(19) United States
(12) Patent Application Publication
(10) Pub. No.: US 2007/0183976 A1
(43) Pub. Date: Aug. 9, 2007

(54) Composition for ultrasound therapy and pharmaceutical liquid composition containing the same

(75) Inventor: Evan C. Unger, Tucson, AZ (US)

Correspondence Address: DLA PIPER US LLP, 4365 EXECUTIVE DRIVE, SUITE 1100, SAN DIEGO, CA, 92121-2133, US

(73) Assignee: IMARX THERAPEUTICS, INC., Tucson, AZ (US)

(21) Appl. No.: 11/649,971

(22) Filed: Jan. 5, 2007

Related U.S. Application Data
(60) Provisional application No. 60/757,114, filed on Jan. 6, 2006.

(51) Int. Cl. A61K 49/04; A61K 49/22

(52) U.S. Cl. 424/9.4; 424/9.52

(57) ABSTRACT

The present invention provides pharmaceutical liquid compositions, comprising a liquid, a prodrug, and a large quantity of microbubbles formed by a gas dispersed in the liquid, the microbubbles having a diameter between about 0.1 μm and 100 μm, wherein a majority of the prodrug is present outside the microbubbles. The compositions can be used for an ultrasound therapy or for enhancing effects of an ultrasound therapy. The compositions are also useful for enhancing the therapeutic effect of a prodrug, and for enhancing the therapeutic effect of ultrasound.

### CROSS-REFERENCE TO RELATED APPLICATIONS

This application claims priority under 35 U.S.C. § 119(e) to U.S. Patent Application Ser. No. 60/757,114, filed Jan. 6, 2006, which is herein incorporated by reference in its entirety.

### BACKGROUND

1. Field of the Invention

The present invention relates generally to the field of ultrasound therapy, such as to booster useful for enhancing the effects of ultrasound in the therapy of various diseases. More particularly, it relates to a booster comprising microbubbles, a pharmaceutical liquid composition comprising such a booster, and the use thereof in the ultrasound therapy of various diseases.

2. Background Information

It is known that some diseases can be remedied by the aid of ultrasonic vibration. For example, it is described in Japanese patent document No, 115591/1977 (Kokai) that percutaneous absorption of a medicament is enhanced by applying a ultrasonic vibration. Japanese patent document No. 180275/1990 (Kokai) discloses a drug-injecting device which is effective on the diffusion and penetration of the drug by applying a ultrasonic vibration in the step of injecting a drug into a human body via a catheter or a drug-injecting tube. U.S. Pat. Nos. 4,953,565 and 5,007,438 also disclose the technique of percutaneous absorption of medicaments by the aid of ultrasonic vibration. It was also reported that a tumor can be remedied by concentratedly applying ultrasound from outside the body.

Furthermore, U.S. Pat. No. Re. 36,939 offers a booster useful for enhancing the effects of ultrasound in the therapy of various diseases and a pharmaceutical liquid composition containing the booster and a medicament which shows enhanced diffusion and penetration of the medicament into the body by applying ultrasound. The '939 patent discloses using specific anti-thrombosis agents (i.e., urokinase, tissue plasminogen activator, etc.); however, the '939 patent is silent with respect to using a prodrug and does not describe the numerous advantageous aspects of using a prodrug as a booster useful for enhancing the effects of ultrasound in the therapy of various diseases.

In order to enhance the therapeutic effects with ultrasound, it is typically necessary to apply a higher energy of a ultrasonic vibration. However, if the energy of a ultrasonic vibration is too high, it can cause various undesirable effects, such as burns or unnecessary heat. On the other hand, when the energy of a ultrasonic vibration is lowered for eliminating such disadvantages, the effectiveness of the ultrasound treatment is reduced. Accordingly, better methods of ultrasound therapy are desired.

### SUMMARY

According to one embodiment of the present invention, a pharmaceutical liquid composition useful for an ultrasound therapy or for enhancing effects of an ultrasound therapy is provided, the composition comprising a liquid, a prodrug; and a plurality of microbubbles formed by a gas dispersed in the liquid, the microbubbles having a diameter between about 0.1 μm and 100 μm, wherein a majority of the prodrug is present outside the microbubbles.

According to other embodiments of the present invention, such a pharmaceutical liquid composition is useful for enhancing the therapeutic effect of a prodrug, for enhancing the therapeutic effect of ultrasound, and for dosing a subject with a pharmaceutical preparation.

### BRIEF DESCRIPTION OF THE DRAWINGS

FIG. 1 shows a schematic view of one of the micro-bubbles contained in the booster of the invention.

FIG. 2 shows a schematic sectional view of one embodiment of a drug administration device used for injecting, pouring, applying or circulating the booster or the pharmaceutical liquid composition of the invention.

FIG. 3 shows a schematic sectional view of one embodiment of a drug administration device used for percutaneous injection of the booster or the pharmaceutical liquid composition of the invention

FIG. 4 and FIG. 5 show graphs showing fibrinolysis by application of ultrasound with or without the booster of the invention.

### DETAILED DESCRIPTION

The present invention relates ultrasound therapy. More specifically, some embodiments of the invention provide a booster described in more detail below. The booster is useful for enhancing the effects of ultrasound in the therapy of various diseases. Other embodiments of the invention provide a pharmaceutical liquid composition containing the booster and a prodrug. By applying ultrasound to such pharmaceutical liquid composition can enhance diffusion and penetration of the prodrug into the body.

In some embodiments of the invention, the booster comprises a liquid containing gaseous microbubbles having a diameter between about 0.1 μm and 100 μm. The microbubbles are formed by entrapping microspheres of a gas into a liquid. The booster can contain a large quantity of microbubbles. In one exemplary embodiment, the concentration of microbubbles is about 4×10⁷ of the microbubbles per one milliliter of a liquid.

A variety of gases can be employed for fabricating microbubbles. Examples of gases that can be used include air, oxygen, carbon dioxide, and inert gases (e.g. xenon, krypton, argon, neon, helium, etc.). The liquid used in the booster includes any liquid which can form microbubbles, for example, human serum albumin (e.g. 3 to 5% human serum albumin), a physiological saline solution, a 5% aqueous glucose solution, an aqueous indocyanine green solution, autoblood, an aqueous solution of maglumine diatriazoate (renografin), or any other liquid X-ray contrast medium.

The booster can be prepared by any method known to those having ordinary skill in the art, for example, by agitating an above-described liquid while blowing an the above-described gas into the liquid. Alternatively a liquid can be to exposed to ultrasound with a sonicator under a gaseous atmosphere, whereby a vibration is given to the liquid to form microbubbles of the gas.

The above-mentioned pharmaceutical liquid composition of the invention comprises a large quantity of microbubbles of a gas and a prodrug in a liquid. According to embodiments of the invention, in the above-mentioned pharmaceutical liquid composition a majority of the prodrug is present outside the microbubbles. The term “majority” in the context of the invention is defined to refer to a portion of the prodrug that is larger than 50% of the total quantity of the prodrug (by mass).

For the purposes of the present invention, a prodrug includes a pharmacological substance (drug) which is administered in an inactive (or significantly less active) form, as these terms are understood by those having ordinary skill in the art. Once administered, the prodrug is metabolized in vivo into the active compound. The microbubbles utilized in the pharmaceutical liquid composition are the same as mentioned above.

The prodrug that can be used in the pharmaceutical liquid composition includes any known prodrugs effective for the desired therapy which can be absorbed percutaneously, for example, pro-urokinase and the like. The prodrug can be contained in a therapeutically effective amount as commonly used.

The pharmaceutical liquid composition can be prepared by mixing a prodrug with an above-describe booster containing microbubbles of a gas in a liquid, to obtain a pharmaceutical liquid composition a majority of the prodrug is present outside the microbubbles. In some embodiments, not only a majority of the prodrug is present outside the microbubbles, but also a majority of the prodrug is not incorporated in the shell of the microbubble.

The mixing ratio can vary depending on the desired amount and kind of the prodrug and the kind of the liquid, but is typically in a range of between about 1:100 and 100:1 (by mass) of a prodrug to a booster.

According to embodiments of the invention, the ultrasonic therapeutic is boosted by the presence of a booster. Particularly, when a pharmaceutical liquid composition containing the booster and a prodrug is poured or injected into a body in parenteral routes, such as intravenously, percutaneously or intramuscularly, while applying thereto a ultrasonic vibration, the therapeutic effects of the prodrug is enhanced. When an ultrasound from a ultrasonic element is applied to the liquid containing the booster and prodrug, cavitation occurs in the liquid composition, and the prodrug is diffused and penetrated into the desired portion of the biological body by the aid of vibration induced by the cavitation.

The cavitation occurs when the level of vibration energy exceeds a certain threshold value. When the ultrasound is applied to the liquid composition of the invention, the threshold value of the vibration energy lowers due to the presence of a large quantity of microbubbles of a gas. Accordingly, the microbubbles of a gas act as nucleus of cavitation and thereby the cavitation is facilitated. Therefore, according to embodiments of the invention, the desired ultrasonic energy necessary for the desired diffusion and penetration of a prodrug is achieved by less energy of ultrasonic vibration energy than without use of microbubbles.

Those ordinarily skilled in the art will select ultrasound having the desired properties. For example, the ultrasound can be used as generated by conventional ultrasonic devices which can supply a ultrasonic signal of 20 KHz to several MHz.

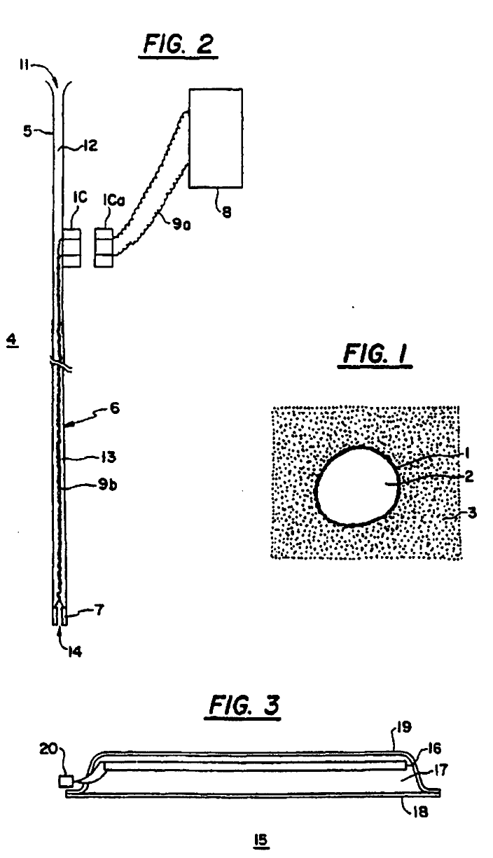

Now, with reference to the accompanying drawings, the invention is illustrated in more detail as follows. FIG. 1 shows a schematic view of one of the microbubbles contained in the booster of the invention, wherein the microbubble has a diameter between about 0.1 μm and 100 μm and is composed of a shell of human serum albumin 1 and gas 2 entrapped within the shell 1. The microbubble is contained in a liquid 3, such as 5% human serum albumin solution. An exemplary concentration of microbubbles can be above 4×10⁷ microbubbles per milliliter.

The booster is mixed with a prodrug to form a pharmaceutical liquid composition in which a majority of the prodrug is present outside the microbubbles. The pharmaceutical liquid composition is directly administered to the diseased part with an appropriate device, for example, with an exemplary drug administration device 4 shown on FIG. 2. The drug administration device 4 comprises a base tube 5, to which the pharmaceutical liquid composition is supplied, and an end tube 6 which is to be inserted into the tissue of the biological body and through which the pharmaceutical liquid composition administered into the diseased part, for example, by pouring or injecting.

The end tube 6 of the drug administration device 4 is provided with an ultrasonic element 7, such as a cylindrical ceramic oscillator. An ultrasonic signal of between about 20 kHz and several megahertz is directed to the ultrasonic element from an ultrasonic oscillation circuit 8, via a conductor 9a, connectors 10 and 10a provided on the side of the base tube 5, a part of the base tube 5, and a conductor 9b provided within the end tube 6.

The prodrug is administered from a pharmaceutical liquid composition which is prepared by previously mixing the prodrug with the above-described booster comprising a large quantity of microbubbles. To prepare the pharmaceutical liquid composition, the prodrug and the booster are mixed in a mass ratio of between about 1:100 and 100:1 by weight. The pharmaceutical liquid composition is then poured into the base tube 5 from the supply opening 11 provided on the tip of the base tube 5, passes through a flow path 12 within the base tube 5 and a flow path 13 within the end tube 6, and is then administered to the diseased part or the portion close thereto of the patient via a pouring opening 14 provided at the bottom of the end tube 6.

When the pharmaceutical liquid composition is administered through the pouring opening 14, an ultrasonic energy generated from a ultrasonic element 7 is directed to the liquid composition, causing cavitation to occur. Microbubbles are formed at the occurrence of cavitation and when the microbubbles are decomposed, energy is generated, thus promoting diffusion and penetration of the prodrug into the tissue to be treated. Since the pharmaceutical liquid composition contains a large quantity of microbubbles, the microbubbles act as a nucleus for the cavitation, thereby lowering the threshold value of occurrence of cavitation and thus facilitating the process of cavitation. Accordingly, the cavitation is generated using less energy than the energy that would be needed if no booster is used. It has been found that the cavitation occurs most easily where the liquid contains microbubbles of a gas having a diameter between about 0.1 μm and 100 μm.

The drug administration device 4 shown on FIG. 2 can be used, for example, for administering a pharmaceutical liquid composition into a blood vessel. For instance, in the treatment of coronary thrombosis, a pharmaceutical liquid composition comprising a booster of the invention and a pro-urokinase can be injected into the part of thrombosis or the close portion thereof using the drug administration device 4. In this exemplary embodiment, the tip of the end tube 6 is inserted into the portion close to the thrombosis and ultrasound is applied. As a result, the thrombolytic effects of the prodrug are significantly increased and, furthermore, the blood flow is recovered within a shorter period of time in comparison with the administration of the prodrug without the booster.

The drug administation device 4 can be also used for the removing hematoma when there is bleeding of brain. For example, a pharmaceutical liquid composition comprising a booster of the invention and a thromolytic agent (e.g. pro-urokinase) is administered to the portion of hematoma using the drug administration device 4, and ultrasound is applied, as described above. As a result, the hematoma is easily lysed.

In another embodiment of the invention, the pharmaceutical liquid composition can be administered percutaneously using a drug administration device 15 shown on FIG. 3. In this embodiment, the drug administration device 15 suitable for percutaneous administration of a prodrug, and having a layer of a prodrug 17 is provided below an ultrasonic element 16 (e.g. a disc shaped ceramic oscillator, etc.). The bottom portion of the device includes an adhesive prodrug-permeable layer 18. The device also includes a plastic cover 19. An ultrasonic signal is directed to the ultrasonic element 16 from an ultrasonic oscillation circuit provided outside via a connector 20, like in the drug administration device 4 shown on FIG. 2.

In the device 15 of FIG. 3, a pharmaceutical liquid composition comprising a mixture of a booster and a prodrug is contained in the layer of a prodrug 17. When the device 15 is used, it is attached to the skin using the adhesive layer 18. An ultrasonic signal is then directed to the ultrasonic element 16, giving an ultrasonic vibration to both of the prodrug layer 17 and the skin. As a result, the prodrug contained in the prodrug layer 17 passes through the skin and penetrates into the tissue to be treated. In this embodiment, since microbubbles of a gas are contained in the prodrug layer 17, the cavitation occurs within the prodrug layer 17 as a result of the application of ultrasound. Hence, even though a lower energy of the ultrasonic vibration can be supplied from the ultrasonic element 16, the diffusion and penetration of the prodrug can still be effective leading to rapid absorption of the prodrug.

The booster of the invention can also be used alone in the ultrasound therapy, i.e., without mixing with a prodrug. For example, it can be used in the therapy of tumors by heating the diseased part of the tissue with ultrasound. In this application, by concentratedly applying an ultrasonic vibration outside the biological body, a booster comprising a large quantity of microbubbles is injected into the blood vessel or to the portion close to the diseased part followed by application of ultrasound. As a result, the effect of heating with ultrasound is enhanced and thereby the therapeutic effects are significantly improved. In this embodiment, the process of cavitation is facilitated by the application of ultrasonic vibration due to the use of a liquid containing microbubbles Accordingly, even though a lower energy of the ultrasonic vibration can be supplied from the ultrasonic element 16, the diffusion and penetration of the prodrug can still sufficient, thereby avoiding the undesirable burns and unnecessary heating at other portions of the body.

The above-described treatment of tumors is more effective if used in combination with a chemotherapeutic agent suitable for the treatment of the tumors, by which the effects of the chemotherapeutic agent are more enhanced, where the diffusion and penetration of the prodrug are improved owing to the booster.

The booster of the invention is considered safe. The substance such as human serum albumin in the booster of the invention is easily metabolized within, and excreted outside, the body, and therefore, it is not harmful to human body. The gas trapped within the microbubbles is easily dissolved in the blood fluid and thus removed.

### EXAMPLES

The following examples are provided to further illustrate the advantages and features of the present invention, and as a guide for those skilled in the art, but are not intended to limit the scope of the invention. All products were used according to manufacturer's instructions, and experiments are conducted under standard conditions known to those skilled in the art, unless otherwise specified.

### Example 1

### Preparation of a Booster

8 ml of a 5% human serum albumin aqueous solution was placed in a 10 ml syringe and exposed to ultrasound using a sonicator at a frequency of 20 KHz. As a result, a large quantity of microbubbles of air were formed in the human serum albumin, to form a booster comprising a human serum albumin containing a large quantity of microbubbles of air.

### Example 2

### Preparation of a Pharmaceutical Liquid Composition

The 5% human serum albumin solution containing a large quantity of microbubbles of air prepared as in Example 1 was mixed with urokinase (concentration 1200 IU/ml) to form the desired pharmaceutical liquid composition.

### Example 3

### Forming Artificial Thrombosis

An artificial thrombosis was formed by Chandler's method. A sample of blood (1 ml) collected from a healthy person was entered into a flexible tube (inside diameter 3 mm, length 265 mm) and calcium chloride was added. The tube was shaped like a loop and was rotated at 12 r.p.m. for 20 minutes to give an artificial thrombosis model.

### Example 4

### Fabrication of an Ultrasonic Catheter and Testing

A ceramic ultrasonic element (width 2 mm, length 5 mm, thickness 1 mm) was inserted into the tip of a catheter (diameter 2 mm), and an oscillating element was connected to an oscillator provided outside with a fine connector passed through the catheter. A fine tube for pouring a test solution was provided at an opening opposite to the opening of the catheter end.

The artificial thrombosis prepared as described above was added to a test tube together with blood, and the ultrasonic catheter was inserted into the test tube so that the end of the catheter was set close to the portion of the artificial thrombosis (at a distance of about 5 mm). To the test tube was added a mixture of urokinase and a booster prepared as in Example 1 at a rate of 1 ml per minute, wherein urokinase (concentration 1200 Ill/ml) and the booster were mixed immediately before pouring at a mixing ratio of 1:1 by mass. The mixture was refluxed while keeping the volume of the test solution at a constant level by removing excess volume of the solution by suction.

The mixture was exposed to the ultrasound at a frequency of 170 KHz to by a pulse method (exposure for 2 seconds followed by termination of exposure for 4 seconds) for 2 minutes. Total exposure time was 40 seconds). After the exposure, the ultrasonic catheter was removed from the test tube, and the mixture was incubated at 37° C. for between 5 and 120 minutes, washed with a physiological saline solution several times and dried overnight. Thereafter, the dried mixture was weighed. As a control, the above procedure was repeated by using only a physiological saline solution.

The test results were as follows. The rate of fibrinolysis was calculated by the following equation:
Fibrinolysis rate (%)=(A−B)/A×100, wherein
A is the weight of thrombosis in control; and
B is the weight of thrombosis treated.

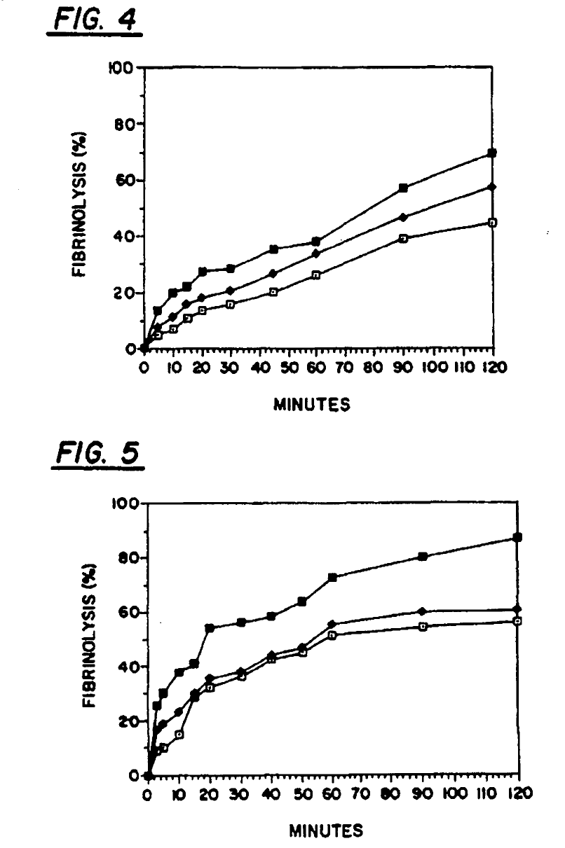

The results (average of two tests) are shown on FIGS. 4 and 5. On these figures, the symbol “−” is for the data obtained in the addition of urokinase alone without exposure of ultrasound. The symbol of solid diamond stands the data obtained in the addition of urokinase alone with exposure of ultrasound, and the symbol of solid box is for the data obtained in the addition of a mixture of urokinase and the booster with exposure of ultrasound. FIG. 4 shows the results in the thrombosis prepared by using blood collected from one person.

As shown on FIG. 4, the time for achieving 20% fibrinolysis was about 45 minutes by urokinase alone without exposure of ultrasound, 30 minutes by a combination of urokinase and exposure of ultrasound, and only 10 minutes by a combination of a mixture of urokinase and a booster and exposure of ultrasound. The fibrinolytic effects of urokinase (both the rate of fibrinolysis and the fibrinlytic time) were significantly enhanced by using a booster with application of ultrasound.

FIG. 5 shows the results in the thrombosis prepared by using blood collected from another person and with reduced energy of ultrasound by 15%. As shown on FIG. 5, the fibrinolytic effects were significantly enhanced by using a mixture of urokinase and the booster. That is, in case of using urokinase alone with exposure of ultrasound, the 50% fibrinolysis was achieved by the treatment for 60 minutes, but in case of using a mixture of urokinase and the booster with exposure of ultrasound, it was reduced to one fourth, i.e., it was achieved by the treatment only for 15 minutes.

Although the foregoing invention has been described in some detail by way of illustration and example for purposes of clarity and understanding, it will be apparent to those of ordinary skill in the art in light of the teaching of this invention that certain changes and modifications may be made thereto without departing from the spirit or scope of the claims.

What is claimed is:

1. A pharmaceutical liquid composition, comprising:
(a) a liquid;
(b) a prodrug; and
(c) a plurality of microbubbles formed by a gas dispersed in the liquid, the microbubbles having a diameter between about 0.1 μm and 100 μm,
wherein a majority of the prodrug is present outside the microbubbles, and wherein the composition is used for an ultrasound therapy or for enhancing effects of an ultrasound therapy.

2. The pharmaceutical liquid composition of claim 1, wherein the gas is selected from a group consisting of air, oxygen, carbon dioxide, xenon, krypton, argon, neon, helium, and a combination thereof.

3. The pharmaceutical liquid composition of claim 1, wherein the liquid is a liquid X-ray contrast medium.

4. The pharmaceutical liquid composition of claim 1, wherein the liquid is selected from a group consisting of a 3 to 5% aqueous human serum albumin solution, a physiological saline solution, a 5% aqueous glucose solution, an aqueous indocyanine green solution, autoblood, an aqueous solution of maglumine diatriazoate, and a combination thereof.

5. The pharmaceutical liquid composition of claim 1, wherein the liquid is a 3 to 5% aqueous human serum albumin solution.

6. The pharmaceutical liquid composition of claim 1, wherein the prodrug comprises pro-urokinase.

7. A method for enhancing the therapeutic effect of a prodrug, the method comprising:
(a) administering the pharmaceutical liquid composition of claim 1 to a patient in need of treatment; and
(b) applying ultrasound to the pharmaceutical liquid composition, thereby enhancing the therapeutic effect of the prodrug.

8. The method of claim 7, wherein the pharmaceutical liquid composition comprises about 4×10⁷ microbubbles per milliliter.

9. The method of claim 7, wherein the gas is selected from a group consisting of air, oxygen, carbon dioxide, xenon, krypton, argon, neon, helium, and a combination thereof.

10. The method of claim 7, wherein the liquid is a liquid X-ray contrast medium.

11. The method of claim 7, wherein the liquid is selected from a group consisting of a 3 to 5% aqueous human serum albumin solution, a physiological saline solution, a 5% aqueous glucose solution, an aqueous indocyanine green solution, autoblood, an aqueous solution of maglumine diatriazoate, and a combination thereof.

12. The method of claim 7, wherein the liquid is a 3 to 5% aqueous human serum albumin solution.

13. The method of claim 7, wherein the prodrug comprises pro-urokinase.

14. A method of dosing a subject with a pharmaceutical preparation, the method comprising:
(a) administering the pharmaceutical liquid composition of claim I to a patient in need of treatment; and
(b) applying ultrasound to the pharmaceutical liquid composition, thereby dosing the subject with the pharmaceutical preparation.

15. The method of claim 14, wherein the pharmaceutical liquid composition comprises about 4×10⁷ microbubbles per milliliter.

16. The method of claim 14, wherein the gas is selected from a group consisting of air, oxygen, carbon dioxide, xenon, krypton, argon, neon, helium, and a combination thereof.

17. The method of claim 14, wherein the liquid is a liquid X-ray contrast medium.

18. The method of claim 14, wherein the liquid is selected from a group consisting of a 3 to 5% aqueous human serum albumin solution, a physiological saline solution, a 5% aqueous glucose solution, an aqueous indocyanine green solution, autoblood, an aqueous solution of maglumine diatriazoate, and a combination thereof.

19. The method of claim 14, wherein the liquid is a 3 to 5% aqueous human serum albumin solution.

20. The method of claim 14, wherein the prodrug comprises pro-urokinase.

21. A composition for enhancing the effects of ultrasound in the therapy of diseases, the composition comprising:
(a) a liquid;
(b) a prodrug; and
(c) a plurality of microbubbles formed by a gas dispersed in the liquid, the microbubbles having a diameter between about 0.1 μm and 100 μm,
wherein a majority of the prodrug is present outside the microbubbles and is not incorporated in the shell.

22. The composition of claim 21, wherein the ultrasound is administered in conjunction with the prodrug.

23. A method for enhancing the therapeutic effect of ultrasound comprising:
(a) administering the pharmaceutical liquid composition of claim 1 to a patient in need of treatment; and
(b) applying ultrasound to the pharmaceutical liquid composition, thereby enhancing the therapeutic effect of ultrasound.

24. The method of claim 23, wherein the pharmaceutical liquid composition is administered in proximity to a diseased part.

25. The method of claim 23, wherein the pharmaceutical liquid composition is injected into a blood vessel near a diseased part.

26. A method for enhancing the therapeutic effect of a prodrug administered to a patient, the method comprising:
(a) administering the prodrug to the patient;
(b) administering to the patient a liquid composition comprising a plurality of microbubbles formed by a gas dispersed in the liquid, the microbubbles having a diameter between about 0.1 μm and 100 μm;
(b) applying ultrasound to the liquid composition and the prodrug,
wherein a majority of the prodrug is present outside of the microbubbles, thereby enhancing the therapeutic effect of the prodrug.

27. The method of claim 26, wherein the liquid composition is injected into a blood vessel near a diseased part.

28. A method of enhancing the therapeutic effect of ultrasound, comprising;
(a) in a liquid containing a prodrug, fabricating microbubbles formed by a gas dispersed in the liquid, the microbubbles having a diameter between about 0.1 μm and 100 μm, wherein a majority of the prodrug is present outside of the microbubbles;
(b) administering the liquid to a patient in need of treatment; and
(b) applying ultrasound to the microbubbles,
thereby enhancing the therapeutic effect of ultrasound.

29. The method of claim 28, wherein the liquid is administered in the proximity of a diseased part.

30. The method of claim 28, wherein the liquid is injected in a blood vessel near the diseased part.
